(19) United States
(12) Patent Application Publication
(10) Pub. No.: US 2013/0103090 A1
(43) Pub. Date: Apr. 25, 2013

(54) VERTEBRAL ROD SYSTEM AND METHODS OF USE

(71) Applicant: Julien Prevost, Memphis, TN (US)

(72) Inventor: Julien Prevost, Memphis, TN (US)

(73) Assignee: WARSAW ORTHOPEDIC, INC., Warsaw, IN (US)

(21) Appl. No.: 13/281,141

(22) Filed: Oct. 25, 2011

(51) Int. Cl. A61B 17/70

(52) U.S. Cl. 606/254

(57) ABSTRACT

A vertebral rod comprises a first elongated rod portion and a second elongated rod portion. A flexible intermediate portion is disposed between the first rod portion and the second rod portion. The intermediate portion includes a wall defining at least one cavity through a surface of the wall. The at least one cavity includes a first cavity portion disposed in a first plane and a second cavity portion disposed in a second plane transverse to the first plane. The at least one cavity defines a first tab having a free end. Methods of use are disclosed.

### TECHNICAL FIELD

The present disclosure generally relates to medical devices for the treatment of spinal disorders, and more particularly to a dynamic vertebral rod system that provides stability while reducing stress on spinal elements.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders may include discectomy, laminectomy, fusion and implantable prosthetics. During surgical treatment, one or more connecting elements may be attached via fasteners to the exterior of two or more vertebral members. This disclosure describes an improvement over these prior art technologies.

### SUMMARY

Accordingly, a dynamic vertebral rod system is provided that provides stability while reducing stress on spinal elements. Desirably, the vertebral rod system includes a resistance member that provides resistance to motion and stress on the vertebral rod system.

In one embodiment, the vertebral rod comprises a first elongated rod portion and a second elongated rod portion. A flexible intermediate portion is disposed between the first rod portion and the second rod portion. The intermediate portion includes a wall defining at least one cavity through a surface of the wall. The at least one cavity includes a first cavity portion disposed in a first plane and a second cavity portion disposed in a second plane transverse to the first plane. The at least one cavity defines a first tab having a free end.

In one embodiment, the vertebral rod comprises a first elongated rod portion and a second elongated rod portion. A flexible intermediate portion is disposed between the first rod portion and the second rod portion. The intermediate portion includes a wall defining a first cavity through a surface of the wall and a second cavity through the surface of the wall. The first cavity has a first cavity portion disposed in a first plane and a second cavity portion disposed in a plane transverse to the first plane. The first cavity defines a first tab having a free end. The second cavity has a first cavity portion disposed in a second plane and a second cavity portion disposed in a plane transverse to the second plane. The second cavity defines a second tab having a free end.

In one embodiment, a vertebral rod system is provided that comprises a vertebral rod including a first elongated rod portion and a second elongated rod portion. A flexible intermediate portion is disposed between the first rod portion and the second rod portion. The intermediate section has a C-shaped configuration defining a correspondingly shaped inner surface and an open end. The intermediate portion includes a wall defining an outer surface and an inner surface. The intermediate surface further includes a first cavity extending through the wall from the outer surface to the inner surface. The first cavity includes a first cavity portion disposed in a sagittal plane of a body and a second cavity portion disposed in a plane transverse to the sagittal plane. The first cavity defines a first tab having a tree end. A first fastener is configured to fasten the first elongated rod portion to tissue. A second fastener is configured to fasten the second elongated rod portion to tissue. A resistance member is provided having an exterior surface configured for engaging the inner surface of the intermediate portion. The second cavity portion is oriented at a selected transverse angle relative to the first cavity portion to dispose the intermediate section in a flexible configuration and maintain shear resistance of the vertebral rod.

### BRIEF DESCRIPTION OF THE DRAWINGS

The present disclosure will become more readily apparent from the specific description accompanied by the following drawings, in which:

FIG. 1 is a perspective view of one particular embodiment of a vertebral rod of a vertebral rod system in accordance with the principles of the present disclosure;

FIG. 2 is a perspective view of the vertebral rod system with a resistance member disposed with the vertebral rod shown in FIG. 1;

FIG. 3 is a perspective view of the vertebral rod system shown in FIG. 2;

FIG. 4 is a perspective view of the vertebral rod system shown in FIG. 1 attached to vertebrae;

FIG. 5 is a lateral section view of the vertebral rod system and vertebrae shown in FIG 4;

FIG. 6 is a lateral view of the vertebral rod system shown in FIG. 5 illustrating rod movement;

FIG. 7 is a lateral view of the vertebral rod system shown in FIG. 5 illustrating rod movement;

FIG. 8 is a perspective view of one embodiment of the vertebral rod shown in FIG. 1 in accordance with the principles of the present disclosure;

FIG. 9 is a perspective view of one embodiment of the vertebral rod shown in FIG. 1 in accordance with the principles of the present disclosure;

FIG. 10 is a perspective view of one embodiment of the vertebral rod shown in FIG. 1 in accordance with the principles of the present disclosure; and

FIG. 11 is a perspective view of one embodiment of the vertebral rod shown in FIG. 1 in accordance with the principles of the present disclosure.

Like reference numerals indicate similar parts throughout the figures.

### DETAILED DESCRIPTION

The exemplary embodiments of the vertebral rod system and methods of use disclosed are discussed in terms of medical devices for the treatment of spinal disorders and more particularly, in terms of a dynamic vertebral rod system having flexion, extension and torsion capability. It is envisioned that the vertebral rod system and methods of use disclosed provide stability and maintains structural integrity while reducing stress on spinal elements.

In one embodiment, the vertebral rod system includes a rod that partially decouples shear resistance and flexibility, which includes flexion, lateral bending and compression bending characteristics for a dynamic spinal implant application. In one embodiment, the vertebral rod includes at least one partial cut in a cross section of the rod to maintain the same shear resistance of the rod while providing flexibility in bending and compression. In one embodiment, the vertebral rod includes at least one cavity oriented in a configuration to follow the orientation of the facet joints in adjacent vertebrae in the sagittal and transverse planes. These various inclination of the cavity can lock the rod in an orientation in high shear force applications. In one embodiment, tabs are provided to resist anterior/posterior shear forces to maintain the same shear resistance during motion in flexion, extension and lateral bending. In one embodiment, the tabs may be separately formed and attached to the rod.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is contemplated that the disclosed vertebral rod system may be alternatively employed in a surgical treatment with a patient in a prone or supine position, and/or employ various surgical approaches to the spine, including anterior, posterior, posterior mid-line, medial, lateral, postero-lateral, and/or antero-lateral approaches, and in other body regions. The present disclosure may also be alternatively employed with procedures for treating the lumbar, cervical, thoracic and pelvic regions of a spinal column. The vertebral rod system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “superior” and “inferior” are relative and used only in the context to the other, and are not necessarily “upper” and “lower”.

Further, as used in the specification and including the appended claims, “treating” or “treatment” of a disease or condition refers to performing a procedure that may include administering one or more drugs to a patient in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The components of the vertebral rod system can be fabricated from biologically acceptable materials suitable for medical applications, including metals, synthetic polymers, ceramics and bone material and/or their composites, depending on the particular application and/or preference of a medical practitioner. For example, the components of the vertebral rod system, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of the vertebral rod system may have material composites, including the above materials, to achieve various desired characteristics such as strength, rigidity, elasticity, compliance, biomechanical performance, durability and radiolucency or imaging preference. The components of the vertebral rod system, individually or collectively, may also be fabricated from a heterogeneous material such as a combination of two or more of the above-described materials. The components of the vertebral rod system may be monolithically formed, integrally connected or include fastening elements and/or instruments, as described herein.

For example, the vertebral rod can be formed of two or more materials. In one embodiment, elongated rod sections can be fabricated from carbon-reinforced PEEK and an intermediate section can be fabricated from PEEK. In one embodiment, elongated rod sections are fabricated from PEEK and an intermediate section is fabricated from carbon-reinforced PEEK. In one embodiment employing a composite material similar to those described, the elongated rod sections can have a cylindrical geometry and the intermediate section can have a rectangular or oblong geometry.

As a further example, a resistance member of the vertebral rod system may be fabricated from materials such as silicone, polyurethane, silicone-polyurethane copolymers, polymeric rubbers, polyolefin rubbers, hydrogels, semi-rigid and rigid materials, and biocompatible materials such as elastomers, rubbers, thermoplastic elastomers, thermoset elastomers, elastomeric composites and plastics.

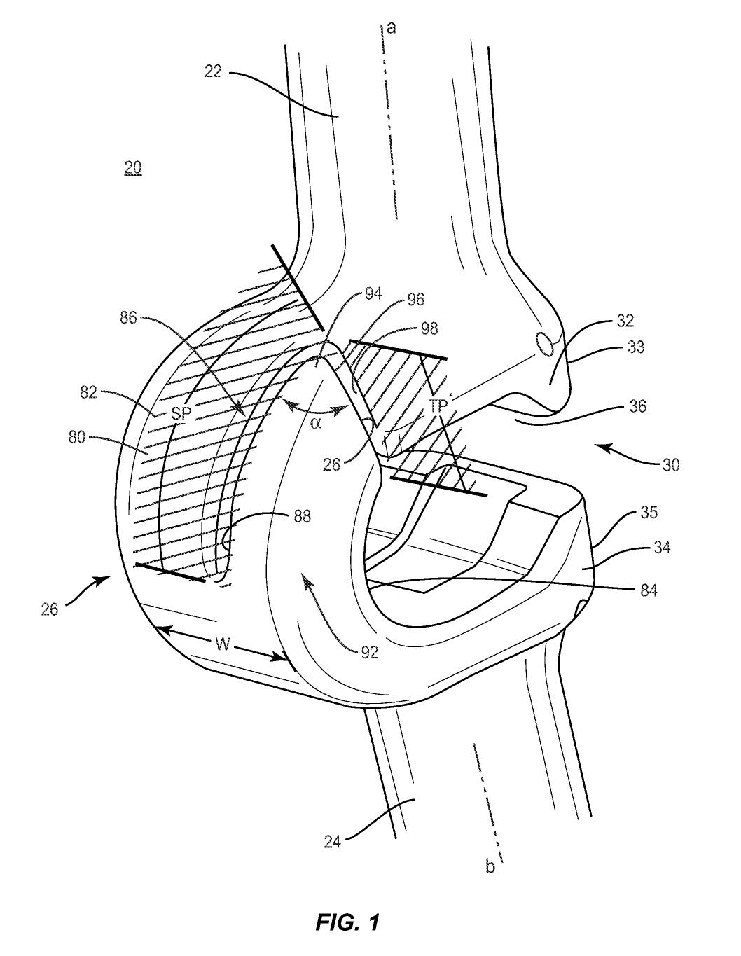

The following discussion includes a description of a vertebral rod system, related components and exemplary methods of employing the vertebral rod system in accordance with the principles of the present disclosure. Alternate embodiments are also disclosed. Reference will now be made in detail to the exemplary embodiments of the present disclosure, which are illustrated in the accompanying figures. Turning now to FIGS. 1-7, there is illustrated components of a vertebral rod system in accordance with the principles of the present disclosure.

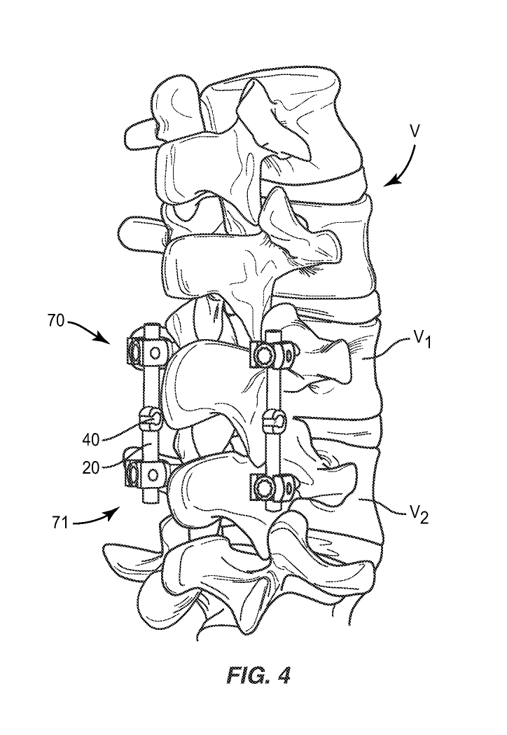

The vertebral rod system is configured for attachment to vertebrae (as shown, for example, in FIG. 4) during surgical treatment of a spinal disorder, examples of which are discussed herein. The vertebral rod system has a vertebral rod 20, which includes a first elongated portion, such as, for example, upper section 22 that defines a longitudinal axis a. A second elongated portion, such as, for example, lower section 24 defines a longitudinal axis b.

An intermediate portion, such as, for example, intermediate section 26 is connected with sections 22, 24 and disposed therebetween as a joining section of the components of vertebral rod 20. It is envisioned that the components of vertebral rod 20 may be monolithically formed, integrally connected or arranged with attaching elements. Intermediate section 26 is flexible relative to sections 22, 24, and is configured to provide resistance to movement of sections 22, 24. It is envisioned that intermediate section 26 may provide increasing, variable, constant and/or decreasing resistance. It is contemplated that sections 22, 24, 26 can be variously dimensioned, for example, with regard to length, width, diameter and thickness. It is further contemplated that the respective cross-section of sections 22, 24, 26 may have various configurations, for example, round, oval, rectangular, irregular, uniform and non-uniform. Section 22 may have a different cross-sectional area, geometry, material or material property such as strength, modulus or flexibility relative to section 24.

Intermediate section 26 includes a wall 80 defining an outer surface 82 and an inner surface 84. Wall 80 has a uniform thickness between surfaces 82, 84 that spans across a width W of intermediate section 26. It is envisioned that wall 80 may have a variable thickness according to the requirements of the particular application.

Intermediate section 26 includes a cavity 86 extending completely through wall 80 from outer surface 82 to inner surface 84. Cavity 86 includes a first cavity portion 88 disposed in a first plane, such as, for example, a sagittal plane SP of a body, such as, for example, a body including vertebrae V (FIG. 4).

Cavity 86 includes a second cavity portion 90 disposed in a plane TP oriented transverse to sagittal plane SP. It is contemplated that planes SP, TP are oriented in alignment along the facet joints of vertebrae V. It is further contemplated that planes SP, TP may be oriented in alignment with other portions of vertebrae V and/or other anatomical structures of a body.

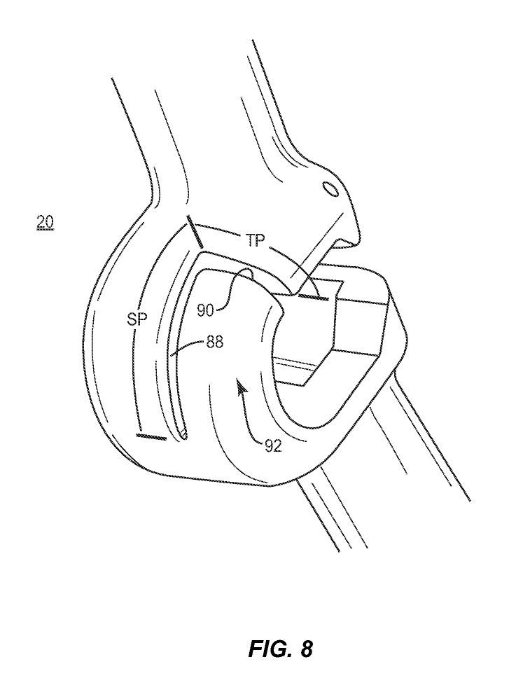

Plane TP is oriented transverse to plane SP at an angle α. It is envisioned that plane TP is oriented relative to plane SP at selected angle α such that intermediate section 26 provides vertebral rod 20 with a configuration to maintain shear resistance of vertebral rod 20 while providing flexibility to vertebral rod 20 during motion of vertebrae V and/or vertebral rod 20. It is further envisioned that such motion may include flexion, extension, torsion, lateral bending and compression bending. For example, angle α is 45 degrees, as shown in FIG. 1. It is contemplated that plane TP is oriented transverse to plane SP at an angle α in a range of 0-60 degrees. Angle α may be acute, obtuse or perpendicular. In one embodiment, as shown in FIG. 8, portion 88 is disposed in sagittal plane SP and portion 90 is disposed in plane TP such that plane TP is oriented transverse to plane SP at angle α equal to 90 degrees. It is contemplated that angle α facilitates orientation of vertebral rod 20 in applications for alignment of vertebral rod 20 with the orientation of a facet of vertebrae V. It is further contemplated that plane TP can be oriented relative to plane SP in various orientations, such as, for example, perpendicular, parallel, co-axial, angularly offset, offset and/or staggered.

In one embodiment, vertebral rod 20 defines a selected angle that indicates the orientation of plane TP relative to a coronal plane of a body. The coronal plane being defined by longitudinal axes a and b. Plane TP is oriented relative to the coronal plane at the selected angle to follow the alignment of the facets, for example, in the thoracic and cervical regions of a spine. In one example, in applications for a lumbar region of the spine, the selected angle is 0 degrees. In one example, in applications for a thoracic region of the spine, the selected angle is 30 degrees. In one example, in applications for a cervical region of the spine, the selected angle is 45 degrees.

Cavity 86 defines a tab 92 having a free end 94. Tab 92 includes an end surface 96 that is spaced apart and opposing an end surface 98 of the remaining portion of wall 80. Free end 94 has a substantially, angularly offset pointed configuration corresponding to the relative angular orientation of angle α. Tab 92 is connected with intermediate section 26 such that the remainder of wall 80 movable relative to tab 92 and end surface 96 is movable relative to end surface 98. Wall 80 and/or other components of vertebral rod 20, for example, sections 22, 26, move about tab 92 during motion of vertebrae V and/or vertebral rod 20. It is contemplated that tab 92 may be monolithically formed, integrally connected or separately attachable with intermediate section 26.

In an unstressed condition of intermediate section 26, tab 92 is disposed in a flush or even alignment with surfaces 82, 84 of the remaining portion of wall 80. End surfaces 96, 98 are aligned and in an opposed facing orientation. In a stressed condition of intermediate section 26, for example, caused by motion of vertebrae V and/or vertebral rod 20, the remaining portion of wall 80 and tab 92, and end surfaces 96, 98 are movable out of alignment such that tab 92 extends beyond surfaces 82, 84. For example, wall 80 is movable about tab 92 such that free end 94 extends outside of surface 82 (FIG. 6) and wall 80 is movable about tab 92 such that free end 94 extends inside of surface 84 (FIG. 7). It is envisioned that end surfaces 96, 98 may be disposed for slidable engagement, frictional engagement and/or non-engagement.

It is envisioned that intermediate section 26 may be configured as a flexible joint having a wide, narrow, round or irregular configuration. It is further envisioned that intermediate section 26 can be variously configured and dimensioned with regard to size, shape, thickness, geometry and material. Intermediate section 26 may also have one or a plurality of elements connecting sections 22, 24 such as spaced apart portions, staggered patterns and mesh. Intermediate section 26 may be fabricated from the same or alternative material to sections 22, 24. Intermediate section 26 may also have a different cross-sectional area, geometry or material property such as strength, modulus and flexibility relative to sections 22, 24. Intermediate section 26 may be connected to sections 22, 24 using various methods and structure including molding of a continuous component, mechanical fastening, adhesive bonding and combinations thereof. It is envisioned that intermediate section 26 has a flexible hinge configuration, which can be offset forward or backward relative to a central axis of rod 20 to modify the flexibility or stiffness of the vertebral rod system. It is further envisioned that particular parameters may be selected to modulate the flexibility or stiffness of the vertebral rod system including the cross-sectional area (or thickness) of intermediate section 26.

Intermediate section 26 has a C-shaped configuration and defines arcuate inner surface 84 and an open end 30. It is contemplated that section 26 may have alternative configurations such as U-shaped, V-shaped or W-shaped. It is further contemplated that vertebral rod 20 may include one or a plurality of intermediate sections 26 spaced along the length of rod 20. In embodiments including a plurality of sections 26, the multiple sections 26 may be disposed in similar, or alternative orientations such as aligned, non-aligned, offset, open end facing or not facing vertebrae and alternate angular orientation.

Upper section 22 is disposed adjacent to an upper portion 32 of open end 30 and the transition defines a front face 33. Lower section 24 is disposed adjacent a lower portion 34 and the transition defines a front face 35. Inner surface 84 defines a cavity 36.

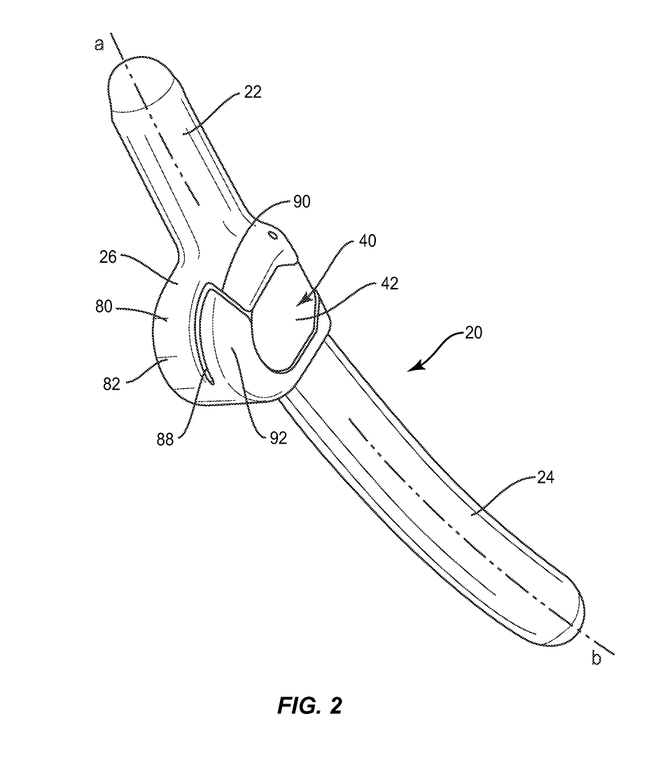

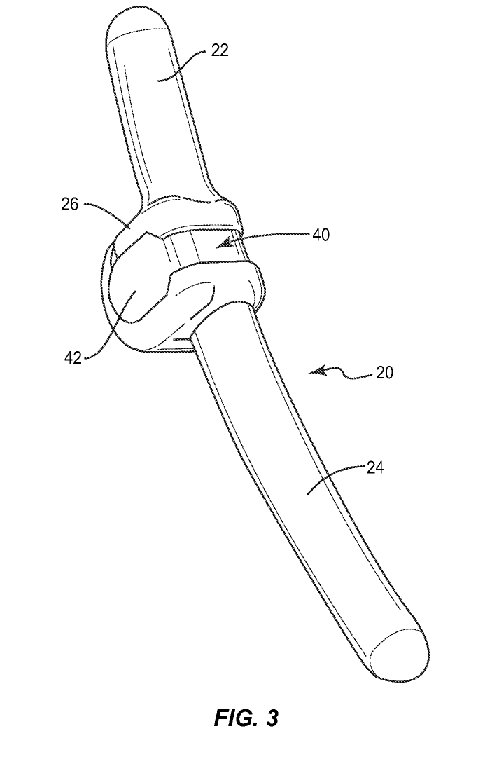

Cavity 36 is configured for disposal of a resistance member, such as, for example, a bumper 40, as shown in FIGS. 2 and 3. Bumper 40 has an exterior surface 42. Bumper 40 is elastic and configured to provide variable resistance to movement of sections 22, 24 and 26. It is contemplated that bumper 40 can provide increasing, variable, constant and/or decreasing resistance. Bumper 40 is disposed within cavity 36 and engages surface 84 in a close fitting engagement. Bumper 40 can be variously configured with regard to size, shape, for example, round, oblong, rectangular, triangular, spherical, and irregular shapes. The material of bumper 40 can be solid or porous, homogeneous or heterogeneous, single polymer or a blend/composite of more than one polymer. It is envisioned that bumper 40 is configured to prevent and/or resist closing of open end 30. It is further envisioned that bumper 40 is secured in place with intermediate section 26, and desirably mechanically secured therewith in a configuration to present migration and expulsion therefrom. In other embodiments, bumper 40 can be textured, encapsulated, adhesively bonded and/or over molded with vertebral rod 20.

In assembly, operation and use, the vertebral rod system is employed with a surgical procedure for treatment of a spinal disorder affecting a section of a spine of a patient, as discussed herein. The vertebral rod system may also be employed with other surgical procedures. In particular, the vertebral rod system is employed with a surgical procedure for treatment of a condition or injury of an affected section of the spine including vertebrae V, as shown in FIGS. 4-7. It is contemplated that the vertebral rod system is attached to vertebrae V for dynamic stabilization of the affected section of the spine to facilitate healing and therapeutic treatment, while providing flexion, extension and torsion capability.

In use, to treat the affected section of the spine, a medical practitioner obtains access to a surgical site including vertebra V in any appropriate manner, such as through incision and retraction of tissues. It is envisioned that the vertebral rod system may be used in any existing surgical method or technique including open surgery, mini-open surgery, minimally invasive surgery and percutaneous surgical implantation, whereby the vertebrae V is accessed through a micro-incision, or sleeve that provides a protected passageway to the area. Once access to the surgical site is obtained, the particular surgical procedure is performed for treating the spinal disorder. The vertebral rod system is then employed to augment the surgical treatment. The vertebral rod system can be delivered or implanted as a pre-assembled device or can be assembled in situ. The vertebral rod system may be completely or partially revised, removed or replaced, for example, replacing bumper 40 only, replacing rod 20 and bumper 40 and using the in-place fastening elements.

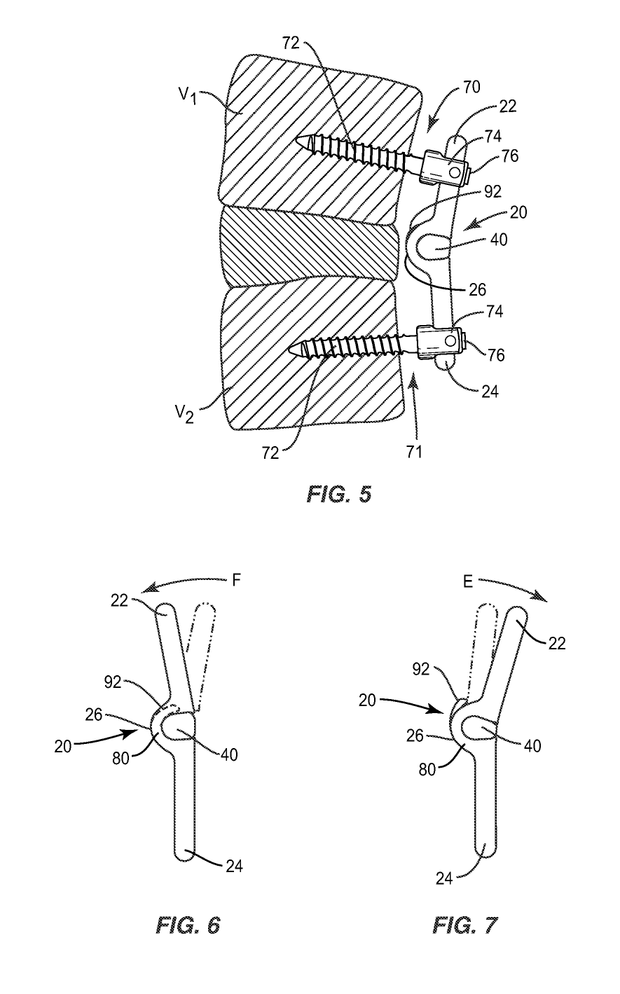

As shown in FIGS. 4-5, a first fastening element, such as, for example, fixation screw assembly 70 is configured to attach upper section 22 to vertebra V₁. A second fastening element, such as, for example, fixation screw assembly 71 is configured to attach lower section 24 to adjacent vertebra V₂. Pilot holes are made in vertebrae V₁, V₂ for receiving fixation screw assemblies 70, 71. Fixation screw assemblies 70, 71 include threaded bone engaging portions 72 that are inserted or otherwise connected to vertebrae V₁, V₂, according to the particular requirements of the surgical treatment. Fixation screw assemblies 70, 71 each have a head 74 with a bore, or through opening and a set screw 76, which is torqued on to sections 22, 24 to attach rod 20 in place with vertebrae V, as will be described.

The vertebral rod system includes two axially aligned and spaced rods 20, with portions of sections 22, 24 extending through the bores of heads 74. Set screws 76 of each head 74 are torqued on the end portions of rods 20 to securely attach rods 20 with vertebrae V₁, V₂. Upon fixation of the vertebral rod system with vertebrae V, vertebral rod 20 is configured to provide increasing resistance to movement of sections 22, 24 during flexion, extension and/or torsion of the spine. For example, vertebral rod 20, as shown in FIG. 5, is in an unloaded state, which corresponds to the unstressed orientation discussed above, where there is no appreciable tensile or compressive loads on vertebrae V₁, V₂. Tab 92 is disposed in a flush or even alignment with surfaces 82, 84 (FIG. 1) of the remaining portion of wall 80. End surfaces 96, 98 are aligned and in an opposed facing orientation. In flexion and/or extension of vertebrae V caused by corresponding movement of the patient, rod 20 reacts with increasing resistance during movement of rod 20 to various orientation(s).

In flexion, as shown in FIG. 6, upper section 22 moves relative to section 24, in the direction of arrow F. Wall 80 flexibly compresses circumferentially about bumper 40. Wall 80 is movable about tab 92 such that free end 94 extends inside of surface 84. This configuration provides flexibility while maintaining resistance during flexion. In extension, as shown in FIG. 7, upper section 22 moves relative to section 24, in the direction shown by arrow E. Wall 80 flexibly expands circumferentially about bumper 40 such that intermediate section 26 compresses bumper 40. Inner surface 84 adjacent bumper 40 is in tension and does not significantly compress bumper 40. Wall 80 moves about tab 92 such that free end 94 extends outside of surface 82. This configuration provides flexibility while maintaining resistance during flexion. The resistance during flexion and extension provides limited movement of vertebrae V for dynamic stabilization of the treated area of the spine.

The vertebral rod system can be used with various bone screws, pedicle screws or multi-axial screws (MAS) used in spinal surgery. It is contemplated that the vertebral rod system may be used with pedicle screws coated with an osteoconductive material such as hydroxyapatite and/or osteoconductive agent such as a bone morphogenic protein for enhanced bony fixation to facilitate motion of the treated spinal area. Rod 20 and bumper 40 can be made of radiolucent materials such as polymers. Radiomarkers may be included for identification under x-ray, fluoroscopy, CT or other imaging techniques. Metallic or ceramic radiomarkers, such as tantalum beads, tantalum pins, titanium pins, titanium endcaps and platinum wires can be used, such as being disposed at the end portions of rod 20 and/or along the length thereof adjacent intermediate section 26 or with bumper 40.

In one embodiment, lower section 24 has an arcuate configuration and an increased length providing the ability to extend over two or more intervertebral elements. It is contemplated that the configuration of the vertebral rod system may provide dynamic or flexible stabilization over a plurality of intervertebral levels, including treated and untreated vertebral and intervertebral levels. It is further contemplated that lower section 24 provides a less flexible, or more rigid stabilization relative to upper section 22 and intermediate section 26. It is envisioned that lower section 24 may be attached with vertebrae across lower lumbar levels such as levels L5-S1. Lower section 24 may be cut or trimmed during a surgical procedure such that the size of vertebral rod 20 can be modified according to patient needs or the particular requirements of a surgical treatment or medical practitioner.

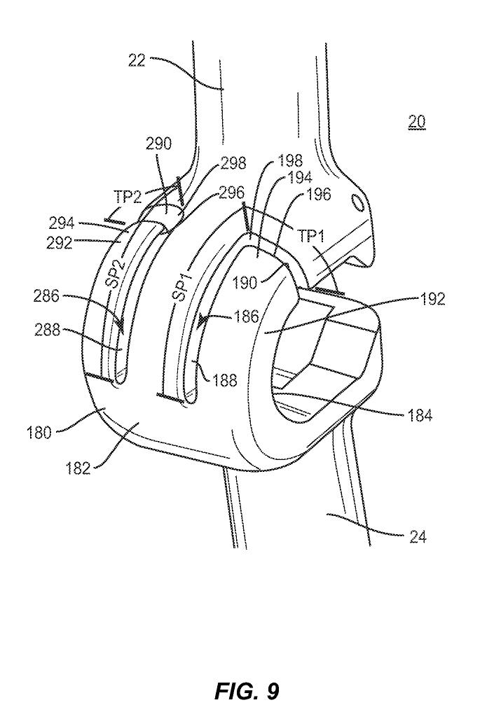

Referring to FIG. 9, in one embodiment, vertebral rod 20, similar to that described with regard to FIGS. 1-7, includes an intermediate section 126, similar to intermediate section 26, connected with sections 22, 24 and disposed therebetween as a joining section of the components of vertebral rod 20.

Intermediate section 126 includes a wall 180 defining an outer surface 182 and an inner surface 184. Intermediate section 26 includes a first cavity 186 extending completely through wall 180 from outer surface 182 to inner surface 184. First cavity 186 includes a first cavity portion 188 disposed in a plane, such as, for example, a sagittal plane SP1.

First cavity 186 includes a second cavity portion 190 disposed in a plane TP1 oriented transverse to sagittal plane SP1, and defines a tab 192 having a free end 194, similar to that shown and described with regard to FIGS. 1-7. Tab 192 includes an end surface 196 that is spaced apart and opposing an end surface 198 of the remaining portion of wall 180. Tab 192 is connected with intermediate section 126 such that the remainder of wall 180 is movable relative to tab 192 and end surface 196 is movable relative to end surface 198. Wall 180 and/or other components of vertebral rod 20, for example, sections 22, 126, move about tab 192 during motion of vertebrae V and/or vertebral rod 20.

Intermediate section 126 includes a second cavity 286 extending completely through wall 180 from outer surface 182 to inner surface 184. Second cavity 286 is spaced apart from first cavity 186 along wall 180. Second cavity 286 includes a first cavity portion 288 disposed in a plane, such as, for example, a sagittal plane SP2.

Second cavity 286 includes a second cavity portion 290 disposed in a plane TP2 oriented transverse to sagittal plane SP2, and defines a tab 292 having a tree end 294, similar to that shown and described with regard to FIGS. 1-7. Tab 292 includes an end surface 296 that is spaced apart and opposing an end surface 298 of the remaining portion of wall 180. Tab 292 is connected with intermediate section 126 such that the remainder of wall 180 is movable relative to tab 292 and end surface 296 is movable relative to end surface 298. Wall 180 and/or other components of vertebral rod 20, for example, sections 22, 126, move about tab 192 during motion of vertebrae V and/or vertebral rod 20.

In an unstressed condition of intermediate section 126, tabs 192, 292 are disposed in a flush or even alignment with surfaces 182, 184 of the remaining portion of wall 180. End surfaces 196, 198 and 296, 298, respectively, are aligned and in an opposed facing orientation. In a stressed condition of intermediate section 126, for example, caused by motion of vertebrae V and/or vertebral rod 20, the remaining portion of wall 180 and tabs 192, 292, and end surfaces 196, 198 and 296, 298 are movable out of alignment such that tabs 192, 292 extend beyond surfaces 182, 184. For example, wall 180 is movable about tabs 192, 292 such that free ends 194, 294 extend outside of surface 182 and wall 180 is movable about tabs 192, 292 such that free ends 194, 294 extend inside of surface 184, similar to the use of rod 20 in flexion and extension, as described above. It is envisioned that tabs 192, 292 may be simultaneously, independently or collectively movable relative to wall 180.

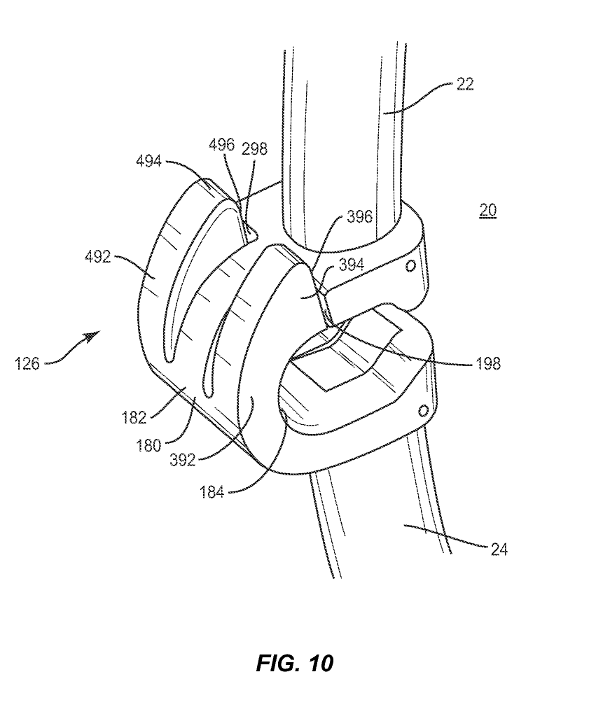

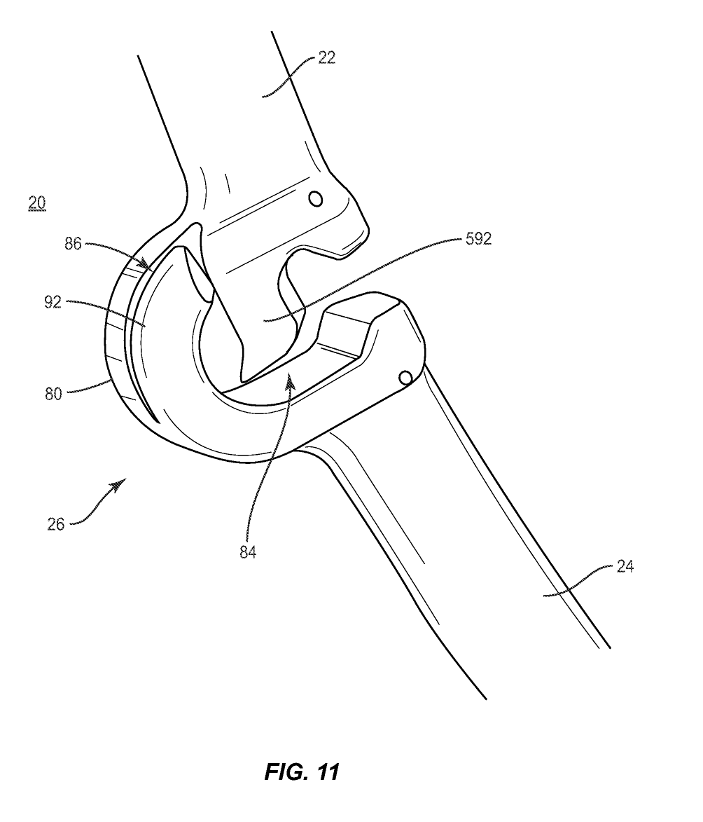

In one embodiment, as shown in FIG. 10, intermediate section 126 includes tabs 392, 492, similar to tabs 192, 292, as defined by the cavities/planes discussed above with regard to FIG. 9 and in relative movement and feature to wall 180. Tabs 392, 492 include extensions 394, 494, respectively, defined with outer surface 182 corresponding to tabs 392, 492. In an unstressed condition of intermediate section 126, the inner surfaces of extensions 392, 492 are disposed in a flush or even alignment with surface 184 of the remaining portion of wall 180. Extensions 394, 494 extend beyond surface 182 of the remaining portion of wall 180 in an unstressed orientation of intermediate section 126.

In a stressed condition of intermediate section 126, extensions 392, 492 include end surfaces 396, 496 that engage end surfaces 198, 298 in a configuration to maintain complete engagement of surfaces 198, 298 during the flexion motion of a spinal segment, and maintain constant shear resistance, for example, a constant contact surface area, during the flexion motion.

Referring to FIG. 11, in one embodiment, intermediate section 26, similar to that described with regard to FIGS. 1-7, includes a second tab 592 extending into interior cavity 84. In a stressed condition of intermediate section 26, the end surfaces of tabs 92, 592 and intermediate section 26 engage in a configuration to maintain complete engagement of the surfaces during the flexion motion of a spinal segment, and maintain constant shear resistance, for example, a constant contact surface area, during the flexion motion.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A vertebral rod comprising:
a first elongated rod portion;
a second elongated rod portion; and
a flexible intermediate portion disposed between the first rod portion and the second rod portion, the intermediate portion including a wall defining at least one cavity through a surface of the wall, the at least one cavity including a first cavity portion disposed in a first plane and a second cavity portion disposed in a second plane transverse to the first plane, the at least one cavity defining a first tab having a free end.

2. A vertebral rod according to claim 1, wherein the at least one cavity includes a first cavity having a first cavity portion disposed in a first plane and a second cavity portion disposed in a second plane transverse to the first plane, and a second cavity having a first cavity portion disposed in a third plane and a second cavity portion disposed in the second plane.

3. A vertebral rod according to claim 2, wherein the third plane is transverse to the second plane.

4. A vertebral rod according to claim 2, wherein the first cavity is spaced apart from the second cavity.

5. A vertebral rod according to claim 2, wherein the first plane is disposed in a first sagittal plane and the third plane is disposed in a second sagittal plane.

6. A vertebral rod according to claim 2, wherein the second cavity defines a second tab having a free end.

7. A vertebral rod according to claim 1, wherein the first plane is disposed in a sagittal plane.

8. A vertebral rod according to claim 1, wherein the first tab extends beyond the surface of the intermediate section in an unstressed orientation of the intermediate section.

9. A vertebral rod according to claim 1, wherein the intermediate section has a C-shaped configuration defining a correspondingly shaped inner surface and an open end.

10. A vertebral rod according to claim 1, wherein the intermediate section defines an inner surface and a resistance member having an exterior surface configured for engaging at least a portion of the inner surface.

11. A vertebral rod according to claim 1, wherein the first tab has a pointed free end.

12. A vertebral rod according to claim 1, wherein the second plane is oriented transverse to the first plane at an angle in a range of 0-60 degrees.

13. A vertebral rod according to claim 1, wherein the second plane is oriented at a selected transverse angle to the first plane in a configuration to maintain shear resistance of the vertebral rod.

14. A vertebral rod according to claim 1, wherein the intermediate section includes an inner surface defining an interior cavity of the intermediate section, the intermediate section further including a second tab extending into the interior cavity.

15. A vertebral rod comprising:
a first elongated rod portion;
a second elongated rod portion; and
a flexible intermediate portion disposed between the first rod portion and the second rod portion, the intermediate portion including a wall defining at a first cavity through a surface of the wall and a second cavity through the surface of the wall, the first cavity having a first cavity portion disposed in a first plane and a second cavity portion disposed in a plane transverse to the first plane, the first cavity defining a first tab having a free end, the second cavity having a first cavity portion disposed in a second plane and a second cavity portion disposed in a plane transverse to the second plane, the second cavity defining a second tab having a free end.

16. A vertebral rod according to claim 15, wherein the first plane is disposed in a first sagittal plane and the second plane is disposed in a second sagittal plane.

17. A vertebral rod according to claim 15, wherein the intermediate section defines an inner surface and a resistance member having an exterior surface configured for engaging at least a portion of the inner surface.

18. A vertebral rod according to claim 15, wherein the first tab and the second tab extend beyond the surface of the intermediate section in an unstressed orientation of the intermediate section.

19. A vertebral rod according to claim 15, wherein the first plane is disposed in parallel relation to the second plane.

20. A vertebral rod system comprising:
a vertebral rod including a first elongated rod portion, a second elongated rod portion, and a flexible intermediate portion disposed between the first rod portion and the second rod portion, the intermediate section having a C-shaped configuration defining a correspondingly shaped inner surface and an open end, the intermediate portion including a wall defining an outer surface and an inner surface, the intermediate portion further including a first cavity extending through the wall from the outer surface to the inner surface, the first cavity including a first cavity portion disposed in a sagittal plane of a body and a second cavity portion disposed in a plane transverse to the sagittal plane, the first cavity defining a first tab having a free end;
a first fastener configured to fasten the first elongated rod portion to tissue;
a second fastener configured to fasten the second elongated rod portion to tissue; and
a resistance member having an exterior surface configured for engaging the inner surface of the intermediate portion,
wherein the second cavity portion is oriented at a selected transverse angle relative to the first cavity portion to dispose the intermediate section in a flexible configuration and maintain shear resistance of the vertebral rod.
